(19) United States
(12) Patent Application Publication
(10) Pub. No.: US 2016/0375176 A1
(43) Pub. Date: Dec. 29, 2016

(54) MINERALIZED COLLAGEN COMPOSITE BONE CEMENTING AND FILLING MATERIAL

(71) Applicant: Beijing Allgens Medical Science and Technology Co., Ltd., Daxing District, Beijing (CN)

(72) Inventors: Zhiye QIU, Beijing (CN); Changming WANG, Beijing (CN); Fuzha CUI, Beijing (CN)

(21) Appl. No.: 15/114,251

(22) PCT Filed: Jan. 24, 2015
(86) PCT No.: PCT/CN2015/071512

§ 371 Date: Jul. 26, 2016

(30) Foreign Application Priority Data
Jan. 28, 2014 (CN) 201410040803.X

(51) Int. Cl. A61L 27/48; A61L 24/06; A61L 24/00; A61L 27/16; A61L 24/02; A61L 27/12; A61L 24/10; A61L 27/24

(52) U.S. Cl. CPC A61L 27/48; A61L 24/102; A61L 24/06; A61L 27/24; A61L 27/16; A61L 24/02; A61L 27/12; A61L 24/0094; A61L 2400/06; A61L 2430/02

(57) ABSTRACT

To cover shortages of existing PMMA bone cement products, including very high hardness and poor biocompatibility, the invention provides a mineralized collagen incorporated PMMA bone adhesive and filling material. Mineralized collagen (MC) is prepared via an in vitro biomimetic mineralization process and has a chemical composition and structure of self-assembled nano-sized calcium phosphate and collagen molecules, thus possessing biomimetic mineralized structure and mechanical properties similar to natural human bone, good biocompatibility, osteogenic activity and biodegradation ability. An MC incorporated bone adhesive and filling material with high compressive strength and low elastic modulus, and improved biocompatibility compared to pure PMMA bone cements may be obtained. Such bone adhesive and filling materials reduce the risk of abrading the host bone tissue and avoiding damage of the implant caused by extrusion, and form osteointegration with the host bone, improving the stability of the bone adhesive and filling materials at the implantation site.

### FIELD OF THE INVENTION

This invention relates to a bone adhesive and filling material in the field of biomedical materials, especially relates to a mineralized collagen incorporated polymethyl methacrylate bone adhesive and filling material.

### BACKGROUND OF THE INVENTION

Polymethyl methacrylate (PMMA), which is also called bone cement, is a commonly used bone adhesive and filling material in orthopedic surgeries. This material has been used for fixation of artificial joint in total hip replacement (THR) and total knee replacement (TKR), as well as in percutaneous vertebroplasty (PVP) and percutaneous kyphoplasty (PKP) for the treatment of compressive vertebral fractures. PMMA bone cements have used for decades in clinics, the safety and immediate effectiveness have been demonstrated by many theoretical studies and clinical practices.

However, complications induced by PMMA bone cements have been reported all over the world. For example, second fractures after PVP surgeries, aseptic loosening of the artificial joint after the implantation, and so on. These complications are caused by mechanical properties and biocompatibilities of PMMA bone cements. The elastic modulus of existing PMMA bone cement is so high that the contacted bone tissue or graft would be abraded or cracked. For example, elastic modulus of PMMA bone cements used in PVP could reach 2˜3 GPa, which is much higher than that of human vertebral cancellous bone (0.05˜0.8 GPa). On the other hand, the biocompatibility of PMMA bone cements derived from their bioinertness. PMMA bone cement cannot for osteointegration with the host bone tissue, result in loosening or even dislodgement of the set bone cement at the implant site. For example, PMMA bone cement may loosening or dislodge from the vertebral body after PVP surgery. Therefore, there are many drawbacks in existing PMMA bone cement products. Complications might be caused by these drawbacks, and has to be revised by a second surgery.

Some studies on the modification of PMMA bone cements have been performed. Hydroxyapatite (HA) and strontium doped HA have been used to improve biocompatibility of PMMA bone cements. However, the incorporation of HA damaged mechanical properties of PMMA bone cements. For example, when the addition of HA reached 20%, the compressive strength of the PMMA bone cement would decrease over 35%, and became lower than the lower limit (70 MPa) of ISO 5833. Some other studies changed monomer component of the PMMA bone cements. For example, N-methyl pyrrolidone (NMP) was added into the monomer liquid of the bone cement to down-regulate elastic modulus of the bone cement. However, the compressive strength was also seriously affected that could not meet clinical requirement. Moreover, the addition of NMP produced risk of lesion on central nervous system, the biosafety of the product needs further evaluations. Bioglass and chitosan were also used to modify PMMA bone cements and got improvement effects on biocompatibility, but the compressive strength of the bone cement decreased to lower than 50 MPa, thus did not in conformity with the requirement of ISO 5833 standard.

Therefore, the clinical available PMMA bone cement products have defects of high elastic modulus and poor biocompatibility, and existing studies only achieved limited improvements to mechanical properties or biocompatibility of PMMA bone cements. Reported solutions cannot provided a modified bone cement with both high compressive strength and low elastic modulus, as well as significantly improved biocompatibility compared to pure PMMA bone cements.

### SUMMARY OF THE INVENTION

In order to cover the shortages of existing PMMA bone cement products, the present invention provides a mineralized collagen incorporated PMMA bone adhesive and filling material. Mineralized collagen (MC) is prepared via an in vitro biomimetic mineralization process. The MC has a chemical composition and structure of self-assembled nano-sized calcium phosphate and collagen molecules, thus possessing biomimetic mineralized structure and mechanical properties similar to the human natural bone, as well as good biocompatibility, osteogenic activity and biodegradation ability. The novel bone adhesive and filling material is prepared by compounding MC and PMMA. The elastic modulus of the set composite should be significantly lower than existing PMMA bone cement products, and the biocompatibility and osteointegration ability should also be better.

The present invention provide a MC incorporated PMMA bone adhesive and filling material, comprising a powder component and a liquid component. The powder component contains MC, prepolymerized PMMA powder and polymerization initiator. The liquid component contains methyl methacrylate (MMA) monomer, polymerization promoter and stabilizer (polymerization inhibitor). The powder component can also contain contrast agent and/or colorant. The liquid component can also contain colorant. The ratio of the said powder component to liquid component is 1.5˜3 g/mL.

In the said powder component,
the content of the MC is 5˜30 wt % of the powder component,
the content of the prepolymerized PMMA powder is 70˜95 wt % of the powder component,
the content of the polymerization initiator is 0.3˜0.8 wt % of the powder component;

In the said liquid component,
the content of the MMA monomer is 98±1 vol % of the liquid component,
the content of the polymerization promoter is 2±1 vol % of the liquid component,
the content of the stabilizer is 10˜100 ppm in the liquid component.

When the said powder component contains contrast agent,
the content of the contrast agent is 5˜40 wt % of the powder component.

The said MC are solid particles of collagen/hydroxyapatite composite with the particle size of 50˜600 μm.

The said MC can also contain calcium phosphate and/or polyester as reinforcement component.

The preparation process of the said MC comprises following steps:

Step S1. Dissolve collagen in any one of hydrochloric acid, nitric acid or acetic acid to form an acidic collagen solution, wherein, the concentration of the collagen is 5.0×10⁻⁵˜5.0×10⁻³ g/mL;

Step S2. Keep stirring the solution obtained by step S1 and add Ca²⁺ containing solution dropwise, wherein, the addition of Ca²⁺ is 0.01˜0.16 mol for 1 g of collagen;

Step S3. Keep stirring the solution obtained by step S2 and add PO₄³⁻ containing solution dropwise, wherein, the molar ratio of the added PO₄³⁻ to the added Ca²⁺ in S2 is Ca/P=1/1˜2/1;

Step S4. Keep stirring the solution obtained by step S3 and add NaOH solution until the pH of the mixture system gets to 6˜8, wherein, precipitation appears when the pH of the mixture system gets to 5˜6, and white suspension will be obtained when the pH gets to 7;

Step S5. Stand the mixture system obtained by step S4 for 24˜120 hours, and then separate out the precipitation and wash it to remove impurity ions, followed by a freeze-drying, the MC powder will be obtained after grinding;

Step S6. Weigh the MC powder obtained by step S5 and fill the powders into a cold compression dies, then compress the dies and make the pressure applied to the composite powders reaches 900˜1200 MPa; maintain the pressure for 30˜300 seconds, and then demould to obtain a dense MC block;

Step S7. Grind the MC block into small particles, and then sieve the particles to screen out MC particles with required particle size.

In the preparation process of the said MC, the step S6 can also be:

Step S6. Weigh the MC powder obtained by step S5, mix the MC powder with calcium phosphate powder and/or polyester powder and fill the mixture powder into a cold compression dies, then compress the dies and make the pressure applied to the composite powder reaches 900˜1200 MPa; maintain the pressure for 30˜300 seconds, meanwhile, heat the dies to make the temperature of the inner materials reach 70˜220° C., and then demould to obtain a dense MC block.

The said calcium phosphate includes hydroxyapatite (HA), α-tricalcium phosphate (α-TCP), β-tricalcium phosphate (β-TCP), octacalcium phosphate (OCP), amorphous calcium phosphate (ACP). The particle size range is 20 nm˜10 μm.

The said polyester includes polylactic acid (PLA), polyglycolic acid (PGA), poly(lactic-co-glycolic acid) (PLGA), polycaprolactone (PCL). The molecular weight is 50,000˜800,000, and the particle size range is 1˜200 μm.

In the said MC/calcium phosphate/polyester composite,
the content of the MC is 10˜75 wt %,
the content of the calcium phosphate is 10˜40 wt %, and
the content of the polyester is 10˜60 wt %.

The molecular weight of the said prepolymerized PMMA powder is 150,000˜600,000, and the particle size is 50˜300 μm.

The said polymerization initiator could initiate free radical polymerization of the MMA to produce PMMA. Preferably, the polymerization initiator is benzoyl peroxide (BPO).

The said contrast agent is an X-ray opaque powder. Preferably, the contrast agent is zirconia (ZrO₂), barium sulfate (BaSO₄) or hydroxyapatite (HA), and the particle size is 0.5˜2 μm.

The said polymerization promoter could oxidize the said polymerization initiator to release free radical, so as to initiate the polymerization of the MMA. Preferably, the polymerization promoter is N,N-dimethyl-p-toluidine (DMPT).

The said stabilizer could inhibit polymerization of the MMA before the application. Preferably, the stabilizer is hydroquinone (HQ).

The said colorant is a liposoluble dye with good biocompatibility, and the dye is soluble in MMA monomer. Preferably, the colorant is chlorophyll (CP).

In the use of the MC incorporated PMMA bone adhesive and filling material, the said powder component and the said liquid component are mixed with a ratio of 1.5˜3 g/mL. After a rapid mixing and waiting for 2˜5 minutes, the mixture could be applied by filling or injecting. A typical working time of the MC incorporated PMMA bone adhesive and filling material is 5˜12 minutes, and a typical setting time is 10˜20 minutes.

For a set product of the MC incorporated PMMA bone adhesive and filling material 24 hours after mixing the powder and liquid components, a typical compressive strength is 70˜100 MPa, a typical compressive modulus is 0.7˜1.5 GPa, a typical bending strength is 40˜60 MPa, and a typical bending modulus is 1.7˜2.5 GPa.

By implementing the invention, a MC incorporated bone adhesive and filling material with high compressive strength and low elastic modulus, as well as improved biocompatibility compared to pure PMMA bone cements could be obtained. On the aspect of mechanical properties, the invented material is more conformity with human bone tissue than the pure PMMA bone cements, thus reducing the risk of abrading the host bone tissue and avoiding the damage of the implant caused by extrusion. On the aspect of biocompatibility, the MC incorporated bone adhesive and filling material contains MC component with good osteogenesis activity, the invented material could therefore form osteointegration with the host bone. The osteointegration is beneficial to the stability of the bone adhesive and filling material at the implantation site, thus making the novel material more safe and reliable. As a result, the MC incorporated bone adhesive and filling material according to the present invention has obvious advantages that are helpful to reduce the complication occurrence of the existing PMMA bone cement products, and has broad application prospects.

### BRIEF DESCRIPTION OF THE DRAWINGS

FIG. 1 is a preparation process flow diagram of the MC according to the present invention;

FIG. 2 is a comparison of the compressive stress-stain curve between the MC incorporated bone adhesive and filling material according to the present invention and a pure PMMA bone cement;

FIG. 3 is a sectional observation of a pure PMMA bone cement;

FIG. 4 is a sectional observation of the bone adhesive and filling material according to the present invention;

FIG. 5 is a scanning electron microscope image of the bone adhesive and filling material according to the present invention;

FIG. 6 is the cell experiment results of the biocompatibility evaluation of the bone adhesive and filling material according to the present invention.

### DETAILED DESCRIPTION OF THE PREFERRED EMBODIMENT

To detailedly explain the present invention, further detail of the present invention will become evident from the attached drawings and operation manners.

FIG. 1 is a preparation process flow diagram of the MC according to the present invention. According to the steps of the FIG. 1, four types of MC particles are prepared by different processes.

### Process 1

### Preparation of Pure MC Particles

Step S1. Dissolve 5 g collagen in 10 L acetic acid solution with the concentration of 0.5 mol/L to form an acidic collagen solution;

Step S2. Keep stirring the solution obtained by step S1 and add 1 L CaCl₂ solution with the concentration of 1 mol/L dropwise;

Step S3. Keep stirring the solution obtained by step S2 and add 1 L Na₂HPO₄ solution with the concentration of 0.6 mol/L dropwise;

Step S4. Keep stirring the solution obtained by step S3 and add NaOH solution until the pH of the mixture system gets to 7;

Step S5. Stand the mixture system obtained by step S4 for 48 hours, and then separate out the precipitation by filtration and wash the precipitation 5 times to remove impurity ions, followed by a freeze-drying, the MC powder will be obtained after grinding;

Step S6. Weigh 6 g MC powder obtained by step S5 and fill the powders into a cold compression dies with the diameter of 11 mm, then compress the dies and make the pressure applied to the composite powders reaches 100 kN, maintain the pressure for 90 seconds, and then demould to obtain a dense MC block;

Step S7. Grind the MC block obtained by step S6 into small particles, and then sieve the particles with stainless steel sieves (mesh size: 50 μm, 200 μm, 300 μm, 400 μm, 500 μm, 600 μm) to screen out MC particles with a series of different particle sizes.

### Process 2

### Preparation of Calcium Phosphate Reinforced MC (MC/CaP)

Steps S1˜S5 are the same to those of above-mentioned in the process 1;

Step S6. Weigh 4.5 g MC powder obtained by step S5, mix the MC powder with 1.5 g HA powder with the particle size of 200 nm˜1 μm, followed by filling the mixture into a cold compression dies with the diameter of 11 mm, then compress the dies and make the pressure applied to the composite powders reaches 110 kN, maintain the pressure for 180 seconds, and then demould to obtain a dense MC block;

Step 7 is the same to that of above-mentioned in the process 1.

### Process 3

### Preparation of Polyester Reinforced MC (MC/PET)

Steps S1˜S5 are the same to those of above-mentioned in the process 1;

Step S6. Weigh 4.5 g MC powder obtained by step S5, mix the MC powder with 1.5 g PLA powder with the particle size of 50˜150 μm, followed by filling the mixture into a cold compression dies with the diameter of 11 mm, then compress the dies and make the pressure applied to the composite powders reaches 65 kN, maintain the pressure for 45 seconds, meanwhile heat the dies to make the temperature of the inside materials reach 180° C., let the dies and the inside materials cool naturally after the pressure maintaining, and then demould to obtain a dense MC block;

Step 7 is the same to that of above-mentioned in the process 1.

### Process 4

### Preparation of Calcium Phosphate/Polyester Reinforced MC (MC/CaP/PET)

Steps S1˜S5 are the same to those of above-mentioned in the process 1;

Step S6. Weigh 4.0 g MC powder obtained by step S5, mix the MC powder with 1.0 g HA powder (particle size: 200 nm˜2 μm) and 1 g PLA powder (particle size: 50˜150 μm), followed by filling the mixture into a cold compression dies with the diameter of 11 mm, then compress the dies and make the pressure applied to the composite powders reaches 75 kN, maintain the pressure for 45 seconds, meanwhile heat the dies to make the temperature of the inside materials reach 200° C., let the dies and the inside materials cool naturally after the pressure maintaining, and then demould to obtain a dense MC block;

Step 7 is the same to that of above-mentioned in the process 1.

TABLE 1
Labels of MC particles prepared by different processes

| | Particle size | | | | |
|---|---|---|---|---|---|
| Process | 50~200 μm | 200~300 μm | 300~400 μm | 400~500 μm | 500~600 μm |
| Process 1 | MC-52 | MC-23 | MC-34 | MC-45 | MC-56 |
| Process 2 | MC/CaP-52 | MC/CaP-23 | MC/CaP-34 | MC/CaP-45 | MC/CaP-56 |
| Process 3 | MC/PET-52 | MC/PET-23 | MC/PET-34 | MC/PET-45 | MC/PET-56 |
| Process 4 | MC/CaP/PET-52 | MC/CaP/PET-23 | MC/CaP/PET-34 | MC/CaP/PET-45 | MC/CaP/PET-56 |

Labels of above MC particles prepared by four different processes are listed in Table 1.

17 groups of MC incorporated PMMA bone adhesive and filling materials were prepared according to Table 2 as the embodiments. All these embodiments were tested on aspects of working properties, mechanical properties and cell culture. 5 samples were used for working property tests for each group, mean values were adopted. For mechanical property tests, 12 samples were used for each group according to ISO 5833, and the results were recorded as M±SD (mean±standard deviation). 5 samples were used for cell culture (MC3T3-E1 cells) for each group.

In contrast, 15 control groups with the powder component according to Table 3 were prepared. These control groups had those free of the MC, those contained lower/higher MC content, and those contained MC with small/large particle size. In the liquid component of these control groups, the MMA monomer were 9.8 mL, DMPT were 0.2 mL, HQ were 40 ppm, and there is no colorant.

TABLE 2
Components of the embodiment groups

| | Powder | | | | | | | |
|---|---|---|---|---|---|---|---|---|
| | | | | Contrast | Liquid | | | |
| No. | MC | PMMA * | BPO | agent ** | MMA | DMPT | HQ | [symbol] |
| E1 | MC-23, 3 g | 15.3 g | 0.1 g | 1.6 g | 9.8 mL | 0.2 mL | 40 ppm | CP |
| E2 | MC-23, 2 g | 11.9 g | 0.1 g | 7.0 g | 9.8 mL | 0.2 mL | 60 ppm | CP |
| E3 | MC-45, 3 g | 15.5 g | 0.8 g | 1.6 g | 9.8 mL | 0.2 mL | 40 ppm | None |
| E4 | MC/CaP-52, 3 g | 15.3 g | 0.1 g | 1.6 g | 9.8 mL | 0.2 mL | 40 ppm | CP |
| E5 | MC/CaP-23, 5 g | 14.9 g | 0.1 g | [symbol] | 9.8 mL | 0.2 mL | 40 ppm | None |
| E6 | MC/CaP-34, 1.5 g | 15.3 g | 0.1 g | 1.6 g | 9.8 mL | 0.2 mL | 40 ppm | None |
| | MC/CaP-45, 1.5 g | | | | | | | |
| E7 | MC/PET-23, 3 g | 15.3 g | 0.1 g | 1.6 g | 9.8 mL | 0.2 mL | 40 ppm | None |
| E8 | MC/PET-34, 2 g | 9.9 g | 0.1 g | 6.0 g | 9.8 mL | 0.2 mL | 80 ppm | None |
| | MC/PET-45, 2 g | | | | | | | |
| E9 | MC/PET-56, 2 g | 16.3 g | 0.12 g | 1.4 g | 9.8 mL | 0.2 mL | 40 ppm | CP |
| E10 | MC/CaP/PET-52, 1.5 g | 15.3 g | 0.1 g | 1.6 g | 9.8 mL | 0.2 mL | 40 ppm | None |
| | MC/CaP/PET-23, 1.5 g | | | | | | | |
| E11 | MC/CaP/PET-34, 3 g | 15.3 g | 0.1 g | 1.6 g | 9.8 mL | 0.2 mL | 40 ppm | None |
| E12 | MC/CaP/PET-34, 4 g | 9.9 g | 0.1 g | 6.0 g | 9.8 mL | 0.2 mL | 80 ppm | CP |
| E13 | MC/CaP/PET-56, 2 g | 16.3 g | 0.12 g | 1.4 g | 9.8 mL | 0.2 mL | 40 ppm | None |
| E14 | MC/CaP/PET-45, 2.75 g | 15.3 g | 0.1 g | [symbol] | 9.8 mL | 0.2 mL | 40 ppm | None |
| | MC/CaP/PET-56, 2.75 g | | | | | | | |
| E15 | MC-34, 2 g | 14.3 g | 0.1 g | 1.6 g | 9.8 mL | 0.2 mL | 40 ppm | None |
| | MC/CaP/PET-34, 2 g | | | | | | | |
| E16 | MC/CaP-23, 1.2 g | 15.3 g | 0.1 g | 1.6 g | 9.8 mL | 0.2 mL | 40 ppm | None |
| | MC/PET-45, 1.8 g | | | | | | | |
| E17 | MC-23, 1 g | 15.3 g | 0.1 g | 1.6 g | 9.8 mL | 0.2 mL | 40 ppm | None |
| | MC/CaP-23, 1 g | | | | | | | |
| | MC/CaP/PET-34, 1 g | | | | | | | |

Notes:
* PMMA was powder with the molecular weight of 300,000~500,000 and particle size of 50~200 μm.
** Contrast agent was ZrO₂ powder with particle size of 1.2 ± 0.4 μm.

TABLE 3
Powder components of the control groups

| | | | | Contrast |
|---|---|---|---|---|
| No. | MC | PMMA * | BPO | agent ** |
| C1 | [symbol] | 18.3 g | 0.1 g | 1.6 g |
| C2 | [symbol] | 15.9 g | 0.1 g | 4.0 g |
| C3 | [symbol] | 13.9 g | 0.1 g | 6.0 g |
| C4 | MC, <50 μm, 3 g | 15.3 g | 0.1 g | 1.6 g |
| C5 | MC-34, 3.5 g | 11.3 g | 0.1 g | 1.6 g |
| | MC-45, 3.5 g | | | |
| C6 | MC-56, 1.5 g | 15.3 g | 0.1 g | 1.6 g |
| | MC, 600~700 μm, 1.5 g | | | |
| C7 | MC/CaP, <50 μm, 5 g | 13.3 g | 0.1 g | 1.6 g |
| C8 | MC/CaP-23, 7 g | 11.3 g | 0.1 g | 1.6 g |
| C9 | MC/PET-34, 8 g | 10.3 g | 0.1 g | 1.6 g |
| C10 | MC/PET-56, 0.5 g | 17.8 g | 0.1 g | 1.6 g |
| C11 | MC/PET, 600~700 μm, 3 g | 15.3 g | 0.1 g | 1.6 g |
| C12 | MC/CaP/PET, <50 μm, 1 g | 16.3 g | 0.1 g | 1.6 g |
| | MC/CaP/PET-52, 1 g | | | |
| C13 | MC/CaP/PET-34, 0.8 g | 17.5 g | 0.1 g | 1.6 g |
| C14 | MC/CaP/PET-45, 7 g | 11.3 g | 0.1 g | 1.6 g |
| C15 | MC/CaP/PET, 600~700 μm, 4 g | 14.3 g | 0.1 g | 1.6 g |

Notes:
* PMMA was powder with the molecular weight of 300,000~500,000 and particle size of 50~200 μm.
** Contrast agent was ZrO₂ powder with particle size of 1.2 ± 0.4 μm.

### Working Property Tests of the Bone Adhesive and Filling Materials

The working properties of the MC incorporated PMMA bone adhesive and filling materials of both embodiment and control groups were tested. Mixing time, waiting time, working time and setting time were recorded. All the tests were performed at 23° C. The results are listed in Table 4.

TABLE 4
Working properties of the bone adhesive and filling
materials according to the present invention

(Unit: second)

| Sample | Mixing | Waiting | Working | Setting |
|---|---|---|---|---|
| No. | time | time | time | time |
| E1 | 30 | 180 | 450 | 900 |
| E2 | 30 | 180 | 450 | 960 |
| E3 | 30 | 270 | 660 | 1200 |
| E4 | 30 | 150 | 360 | 1140 |
| E5 | 30 | 180 | 450 | 900 |
| E6 | 30 | 180 | 450 | 900 |
| E7 | 30 | 180 | 450 | 900 |
| E8 | 30 | 180 | 450 | 1020 |
| E9 | 30 | 120 | 300 | 600 |
| E10 | 30 | 165 | 450 | 1020 |
| E11 | 30 | 180 | 450 | 900 |
| E12 | 30 | 180 | 450 | 1020 |
| E13 | 30 | 120 | 300 | 600 |
| E14 | 30 | 180 | 450 | 900 |
| E15 | 30 | 180 | 450 | 900 |
| E16 | 30 | 180 | 450 | 900 |
| E17 | 30 | 180 | 450 | 900 |
| C1 | 30 | 180 | 450 | 900 |
| C2 | 30 | 180 | 450 | 900 |
| C3 | 30 | 180 | 450 | 900 |
| C4 | Unable to blend | N/A | N/A | N/A |
| C5 | 30 | 180 | 450 | 900 |
| C6 | 30 | 180 | 450 | 900 |
| C7 | Unable to blend | N/A | N/A | N/A |
| C8 | 30 | 180 | 450 | 900 |
| C9 | 30 | 180 | 450 | 900 |
| C10 | 30 | 180 | 450 | 900 |
| C11 | 30 | 180 | 450 | 900 |
| C12 | 30 | 90 | 210 | 1800 |
| C13 | 30 | 180 | 450 | 900 |
| C14 | 30 | 180 | 450 | 900 |
| C15 | 30 | 180 | 450 | 900 |

From the Table 4, the working properties of the MC incorporated PMMA bone adhesive and filling materials according to the present invention were: mixing time was 30 s, waiting time was 2˜5 min, working time was 5˜12 min, and setting time was 10˜20 min. Such working properties were in conformity with clinical operational requirements. These working properties were tested at 23° C. The practical working properties must be determined by actual operating room environment, type of the surgery, habits of the surgeon, surgical instruments, and so on.

It is worth noting that when the particle size of the MC was too small (<50 μm), the beginning blending of the bone adhesive and filling materials would be influenced, thus unable to obtain usable bone adhesive and filling materials (see control groups C4 and C7). This was because that the specific surface area of the small particles were relatively larger than large particles, resulting in a large amount of MMA monomer would be adsorbed in the gap among MC particles, thereby the mobile phase was too few to blend the mixture, and such materials were therefore unavailable.

Even by mixing the undersize MC particles with relatively larger ones together, the waiting time and the working time of the bone adhesive and filling materials were seriously affected. For example, in the control group C12, the MC/CaP/PET particles with <50 μm and 50˜200 ˜m were mixed together, resulting in too short waiting time and working time, thus unsuitable for clinical procedures. Moreover, a large amount of MMA monomer was adsorbed in the gap among small particles, resulting in very long polymerization time. For example, the setting time of the control group C12 was 30 min; otherwise, insufficient polymerization would also be caused, so as to affect the strength of the set bone cement, and the residual monomer in the set bone cement would bring out long-term risk for the patients.

### Mechanical Property Tests of the Bone Adhesive and Filling Materials

Mechanical properties of the bone adhesive and filling materials of both embodiment and control groups were tested according to ISO 5833, including compressive strength, compressive modulus, bending strength and bending modulus. The results are listed in Table 5.

TABLE 5
Mechanical properties of the bone adhesive and filling
materials according to the present invention

(Unit: MPa)

| Sample | Compressive | Compressive | Bending | Bending |
|---|---|---|---|---|
| No. | strength | modulus | strength | modulus |
| E1 | 80.4 ± 7.7 | 780 ± 41 | 42.7 ± 1.6 | 1893 ± 90 |
| E2 | 87.0 ± 5.9 | 1247 ± 36 | 48.6 ± 2.9 | 2024 ± 101 |
| E3 | 82.1 ± 7.3 | 857 ± 34 | 44.5 ± 3.0 | 1906 ± 80 |
| E4 | 74.6 ± 8.9 | 720 ± 43 | 42.9 ± 2.5 | 1850 ± 72 |
| E5 | 81.4 ± 9.7 | 1043 ± 41 | 43.2 ± 2.9 | 1766 ± 69 |
| E6 | 95.0 ± 12.2 | 1354 ± 38 | 54.2 ± 4.5 | 2406 ± 82 |
| E7 | 85.8 ± 7.0 | 1155 ± 52 | 52.0 ± 3.7 | 2258 ± 70 |
| E8 | 86.9 ± 9.5 | 1209 ± 45 | 50.4 ± 2.4 | 2083 ± 105 |
| E9 | 90.4 ± 11.2 | 1367 ± 62 | 55.3 ± 4.5 | 2435 ± 118 |
| E10 | 77.9 ± 10.2 | 909 ± 44 | 46.2 ± 4.2 | 1991 ± 67 |
| E11 | 85.8 ± 9.6 | 1224 ± 57 | 51.8 ± 2.7 | 2174 ± 52 |
| E12 | 88.2 ± 8.4 | 1287 ± 50 | 51.9 ± 3.3 | 2072 ± 120 |
| E13 | 92.8 ± 10.5 | 1358 ± 68 | 57.4 ± 2.6 | 2479 ± 114 |
| E14 | 79.5 ± 8.0 | 1029 ± 42 | 42.9 ± 3.5 | 1919 ± 110 |
| E15 | 84.6 ± 11.2 | 1179 ± 62 | 53.4 ± 2.9 | 2350 ± 106 |
| E16 | 86.3 ± 12.6 | 1287 ± 51 | 53.0 ± 3.4 | 2368 ± 96 |
| E17 | 85.7 ± 9.4 | 1230 ± 54 | 52.2 ± 3.5 | 2314 ± 107 |
| C1 | 105.5 ± 3.2 | 2221 ± 39 | 58.3 ± 2.1 | 2510 ± 39 |
| C2 | 111.5 ± 4.1 | 2308 ± 29 | 59.9 ± 2.9 | 2358 ± 53 |
| C3 | 115.7 ± 3.4 | 2386 ± 50 | 61.0 ± 2.2 | 2009 ± 47 |
| C4 | N/A | N/A | N/A | N/A |
| C5 | 84.5 ± 9.3 | 1250 ± 49 | 37.7 ± 3.6 | 1582 ± 55 |
| C6 | 82.9 ± 7.0 | 1177 ± 62 | 38.2 ± 3.9 | 1628 ± 86 |
| C7 | N/A | N/A | N/A | N/A |
| C8 | 80.7 ± 9.2 | 1189 ± 65 | 38.8 ± 4.2 | 1669 ± 61 |
| C9 | 76.4 ± 11.1 | 803 ± 45 | 38.2 ± 3.5 | 1640 ± 53 |
| C10 | 101.4 ± 3.9 | 1826 ± 42 | 58.1 ± 3.0 | 2488 ± 97 |
| C11 | 86.7 ± 10.0 | 1227 ± 56 | 37.5 ± 2.8 | 1524 ± 61 |
| C12 | 62.8 ± 8.2 | 627 ± 49 | 35.9 ± 4.0 | 1482 ± 63 |
| C13 | 99.3 ± 5.8 | 1740 ± 52 | 56.6 ± 3.1 | 2379 ± 112 |
| C14 | 80.5 ± 6.9 | 837 ± 43 | 38.4 ± 3.9 | 1660 ± 73 |
| C15 | 86.1 ± 6.8 | 1179 ± 54 | 35.8 ± 3.4 | 1383 ± 80 |

It can be seen that the mechanical properties of the MC incorporated PMMA bone adhesive and filling materials were: the compressive strength was 70˜100 MPa, the compressive modulus was 0.7˜1.5 GPa, the bending strength was 40˜60 MPa, and the bending modulus was 1.7˜2.5 GPa. These mechanical properties were in conformity with clinical requirements.

It can be seen from the control groups that:
the set bone cement of the bone adhesive and filling materials without MC (control groups C1˜C3) had very high compressive modulus, thus resulting in high risk of abrasion of the host bone in clinical applications;
when the MC content is too high (more than 30 wt %, control groups C5, C8, C9 and C14), the bending strength of the bone adhesive and filling materials were too low (lower than 40 MPa), thus resulting in high risk of crack of the bone cement;
when the MC content is too low (less than 5 wt %, control groups C10 and C13), the compressive modulus of the bone adhesive and filling materials were still very high, thus resulting in high risk of abrasion of the host bone in clinical applications;
when the particle size of the MC was too large (larger than 600 μm or partially larger than 600 μm, control groups C6, C11, C15), the bending strength of the bone adhesive and filling materials were too low (lower than 40 MPa), thus resulting in high risk of crack of the bone cement;
when the particle size of the MC was too small (smaller than 50 μm or partially smaller than 50 μm, control groups C4, C7, C12), as mentioned above that the control groups C4 and C7 could not be blended, while both of the compressive strength and the bending strength of the control group C12 were too low, thus resulting in high risk of crack of the bone cement;

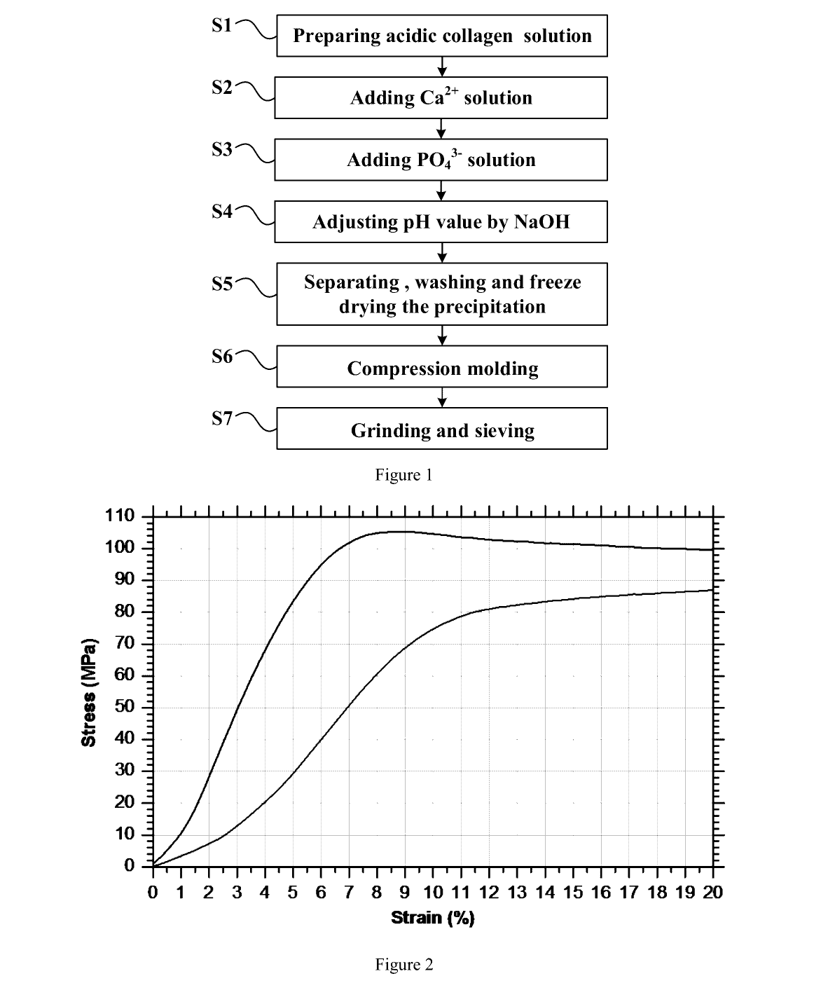

FIG. 2 is a comparison of the compressive stress-stain curve between the MC incorporated bone adhesive and filling material (the embodiment E11) according to the present invention and a pure PMMA bone cement (the control group C1). The shadow is the range of the compressive modulus of normal human vertebral cancellous bone. It can be seen that the slope of the linear section of the stress-strain curve of the set pure PMMA bone cement was relative larger, indicating the pure PMMA bone cement had a large compressive modulus, which was much larger than that range of normal human vertebral cancellous bone. The compressive modulus was significantly down-regulated by the MC, and the curve was generally inside the range of the compressive modulus of normal human vertebral cancellous bone. The mechanical properties were therefore more in conformity with those of human bone, thereby avoiding the damage to the host bone.

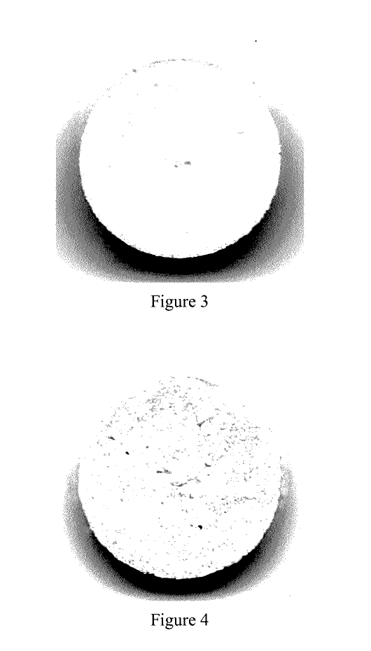

FIG. 3 is a sectional observation of a pure PMMA bone cement (the control group C1); FIG. 4 is a sectional observation of the bone adhesive and filling material (the embodiment E11) according to the present invention. It can be seen that the MC particles were uniformly distributed inside the bone adhesive and filling material.

FIG. 5 is a scanning electron microscope (SEM) image of the bone adhesive and filling material (the embodiment E11) according to the present invention. Energy dispersive X-ray was used to determine the components of MC, PMMA and ZrO₂. It can be seen that the MC and PMMA combined tightly, indicating well compatibility between them that was favorable for maintaining the mechanical strength of the set bone cement.

The biocompatibility of the bone adhesive and filling materials were evaluated by in vitro cell culture experiments. Cells used in the experiments were MC3T3-E1 derived from rat skull. The cells were cultured on the materials in 48-well plates for 7 days, and cells were counted by CCK-8 at the 1^{st}, 3^{rd}, 5^{th} and 7^{th} day. Pure DMEM culture medium was used as the blank control.

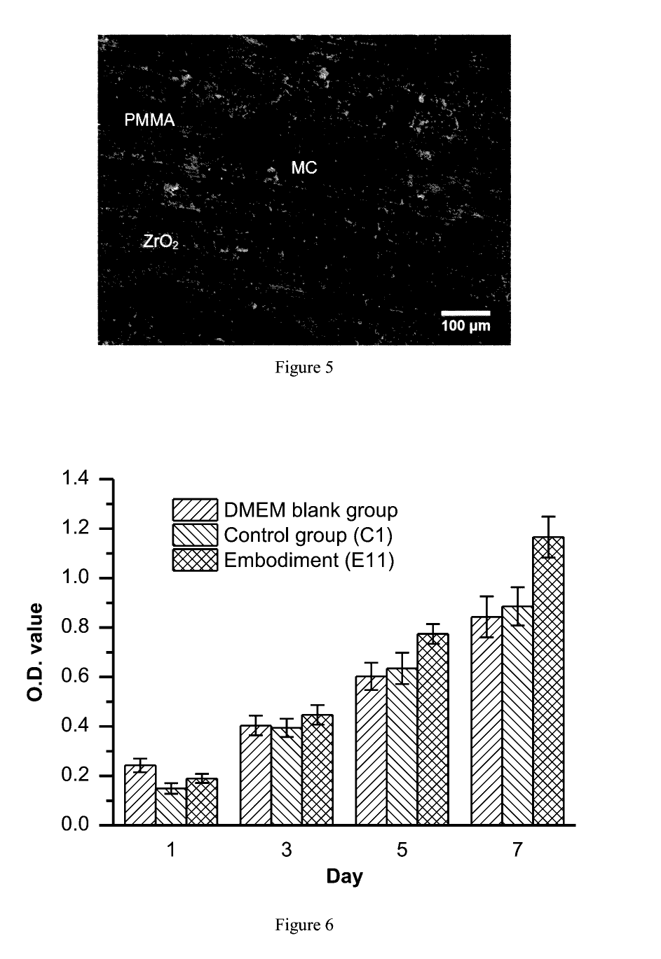

FIG. 6 is the cell experiment results. It can be seen that there were no differences between the pure PMMA (the control group C1) and the blank control groups on cell proliferation. However, cell proliferation on the MC incorporated bone adhesive and filling material (the embodiment E11) was significantly better than that on the pure PMMA and the blank control groups. The results indicated that the biocompatibility of the MC incorporated bone adhesive and filling material is better than the pure PMMA, thus beneficial for the formation of osteointegration with the host bone, thereby preventing loosening and dislodgement of the bone cement at the implanted site.

1. A mineralized collage (MC) incorporated polymethyl methacrylate (PMMA) bone adhesive and filling material, comprising a powder component and a liquid component, the powder component contains mineralized collage (MC), prepolymerized polymethyl methacrylate (PMMA) powder and polymerization initiator, the liquid component contains methyl methacrylate (MMA) monomer, polymerization promoter and stabilizer, the powder component also contains contrast agent and/or colorant, the liquid component also contains colorant, the ratio of the powder component to liquid component is 1.5˜3 g/mL,
in the powder component,
the content of the mineralized collage (MC) is 5˜30 wt % of the powder component,
the content of the prepolymerized polymethyl methacrylate (PMMA) powder is 70˜95 wt % of the powder component,
the content of the polymerization initiator is 0.3˜0.8 wt % of the powder component;
in the liquid component,
the content of the methyl methacrylate (MMA) monomer is 98±1 vol % of the liquid component,
the content of the polymerization promoter is 2±1 vol % of the liquid component,
the content of the stabilizer is 10˜100 ppm in the liquid component,
when the powder component contains contrast agent,
the content of the contrast agent is 5˜40 wt % of the powder component.

2. The mineralized collage (MC) incorporated polymethyl methacrylate (PMMA) bone adhesive and filling material according to claim 1, wherein the mineralized collage (MC) are solid particles of collagen/hydroxyapatite composite with a particle size of 50˜600 μm.

3. The mineralized collage (MC) incorporated polymethyl methacrylate (PMMA) bone adhesive and filling material according to claim 1, wherein the mineralized collage (MC) are solid particles of collagen/hydroxyapatite composite with a particle size of 200˜500 μm.

4. The mineralized collage (MC) incorporated polymethyl methacrylate (PMMA) bone adhesive and filling material according to claim 2, wherein the mineralized collage (MC) also contains calcium phosphate and/or polyester as a reinforcement component.

5. The mineralized collage (MC) incorporated polymethyl methacrylate (PMMA) bone adhesive and filling material according to claim 1, wherein the molecular weight of the prepolymerized polymethyl methacrylate (PMMA) powder is 150,000˜600,000, and the particle size is 50˜300 μm.

6. The mineralized collage (MC) incorporated polymethyl methacrylate (PMMA) bone adhesive and filling material according to claim 1, wherein the molecular weight of the prepolymerized polymethyl methacrylate (PMMA) powder is 300,000˜500,000, and the particle size is 50˜200 μm.

7. The mineralized collage (MC) incorporated polymethyl methacrylate (PMMA) bone adhesive and filling material according to claim 1, wherein the polymerization initiator is able to initiate free radical polymerization of the methyl methacrylate (MMA) to produce polymethyl methacrylate (PMMA), and wherein the polymerization initiator is benzoyl peroxide.

8. The mineralized collage (MC) incorporated polymethyl methacrylate (PMMA) bone adhesive and filling material according to claim 1, wherein the contrast agent is an X-ray opaque powder, wherein the contrast agent is one of zirconia, barium sulfate or hydroxyapatite, and the particle size is 0.5˜2 μm.

9. The mineralized collage (MC) incorporated polymethyl methacrylate (PMMA) bone adhesive and filling material according to claim 1, wherein the polymerization promoter can oxidize the polymerization initiator to release free radicals, so as to initiate the polymerization of the methyl methacrylate (MMA), and wherein the polymerization promoter is N,N-dimethyl-p-toluidine (DMPT).

10. The mineralized collage (MC) incorporated polymethyl methacrylate (PMMA) bone adhesive and filling material according to claim 1, wherein the stabilizer can inhibit polymerization of the methyl methacrylate (MMA) before the application, and wherein the stabilizer is hydroquinone (HQ).

11. The mineralized collage (MC) incorporated polymethyl methacrylate (PMMA) bone adhesive and filling material according to claim 1, wherein in the use of the mineralized collage (MC) incorporated polymethyl methacrylate (PMMA) bone adhesive and filling material, the powder component and the liquid component are mixed with a ratio of 1.5˜3 g/mL, and after a rapid mixing and waiting for 2˜5 minutes, the mixture can be applied by filling or injecting.

12. The mineralized collage (MC) incorporated polymethyl methacrylate (PMMA) bone adhesive and filling material according to claim 1, wherein a typical working time of the mineralized collage (MC) incorporated polymethyl methacrylate (PMMA) bone adhesive and filling material is 5˜12 minutes, and a typical setting time is 10˜20 minutes.

13. The mineralized collage (MC) incorporated polymethyl methacrylate (PMMA) bone adhesive and filling material according to claim 1, wherein for a set product of the mineralized collage (MC) incorporated polymethyl methacrylate (PMMA) bone adhesive and filling material 24 hours after mixing the powder and liquid components, a typical compressive strength is 70˜100 MPa, a typical compressive modulus is 0.7˜1.5 GPa, a typical bending strength is 40˜60 MPa, and a typical bending modulus is 1.7˜2.5 GPa.

14. The mineralized collage (MC) incorporated polymethyl methacrylate (PMMA) bone adhesive and filling material according to claim 2, wherein a preparation process of the mineralized collage (MC) comprises following steps:
step S1, dissolve collagen in any one of hydrochloric acid, nitric acid or acetic acid to form an acidic collagen solution, wherein, the concentration of the collagen is 5.0×10⁻⁵˜5.0×10⁻³ g/mL;
step S2, keep stirring the solution obtained by step S1 and add a Ca²⁺ containing solution dropwise, wherein, the addition of Ca²⁺ is 0.01˜0.16 mol for 1 g of collagen;
step S3, keep stirring the solution obtained by step S2 and add a PO₄³⁻ containing solution dropwise, wherein, the molar ratio of the added PO₄³⁻ to the added Ca²⁺ in S2 is Ca/P=1/1˜2/1;
step S4, keep stirring the solution obtained by step S3 and add an NaOH solution until the pH of a mixture system gets to 6˜8, wherein, precipitation appears when the pH of the mixture system gets to 5˜6, and white suspension will be obtained when the pH gets to 7;
step S5, stand the mixture system obtained by step S4 for 24˜120 hours, and then separate out the precipitation and wash it to remove impurity ions, followed by freeze-drying, a mineralized collage (MC) powder will be obtained after grinding;
step S6, weigh the mineralized collage (MC) powder obtained by step S5 and fill the powder into cold compression dies, then compress the dies and make the pressure applied to the powder reach 900˜1200 MPa; maintain the pressure for 30˜300 seconds, and then demould to obtain a dense mineralized collage (MC) block;
step S7, grind the mineralized collage (MC) block into small particles, and then sieve the particles to screen out mineralized collage (MC) particles with a required particle size.

15. The mineralized collage (MC) incorporated polymethyl methacrylate (PMMA) bone adhesive and filling material according to claim 14, wherein step S6 comprises:
step S6, weigh the mineralized collage (MC) powder obtained by step S5, mix the mineralized collage (MC) powder with calcium phosphate powder and/or polyester powder and fill a mixture powder into cold compression dies, then compress the dies and make the pressure applied to the mixture powder reach 600˜1200 MPa; maintain the pressure for 30˜300 seconds, meanwhile, heat the dies to make the temperature of the inner materials reach 70˜220° C., and then demould to obtain a dense mineralized collage (MC) block.

16. The mineralized collage (MC) incorporated polymethyl methacrylate (PMMA) bone adhesive and filling material according to claim 15, wherein the calcium phosphate includes hydroxyapatite (HA), α-tricalcium phosphate (α-TCP), β-tricalcium phosphate (β-TCP), octacalcium phosphate (OCP), amorphous calcium phosphate (ACP), and the particle size range is 20 nm˜10 μm.

17. The mineralized collage (MC) incorporated polymethyl methacrylate (PMMA) bone adhesive and filling material according to claim 15, wherein the polyester includes polylactic acid (PLA), polyglycolic acid (PGA), poly(lactic-co-glycolic acid) (PLGA), polycaprolactone (PCL), the molecular weight is 50,000˜800,000, and the particle size range is 1˜200 μm.

18. The mineralized collage (MC) incorporated polymethyl methacrylate (PMMA) bone adhesive and filling material according to claim 15, wherein in the mineralized collage (MC)/calcium phosphate/polyester composite,
the content of the mineralized collage (MC) is 10˜75 wt %,
the content of the calcium phosphate is 10˜40 wt %, and
the content of the polyester is 10˜60 wt %.

19. (canceled)
